(19) United States
(12) Patent Application Publication
(10) Pub. No.: US 2023/0111601 A1
(43) Pub. Date: Apr. 13, 2023

(54) ASSESSING ARTIFICIAL INTELLIGENCE TO ASSESS DIFFICULTY LEVEL OF ULTRASOUND EXAMINATIONS

(71) Applicant: University of South Carolina, Columbia, SC (US)

(72) Inventors: Richard Hoppmann, Columbia, SC (US); Floyd Bell, Lexington, SC (US); Robert Haddad, Columbia, SC (US); Keith Barron, Columbia, SC (US)

(73) Assignee: University of South Carolina, Columbia, SC (US)

(21) Appl. No.: 17/963,305

(22) Filed: Oct. 11, 2022

Related U.S. Application Data
(60) Provisional application No. 63/254,207, filed on Oct. 11, 2021.

(51) Int. Cl. G16H 40/20; G06N 3/08; G16H 50/30; G16H 10/60

(52) U.S. Cl. CPC G16H 40/20; G06N 3/08; G16H 50/30; G16H 10/60

(57) ABSTRACT

Described herein are systems and methods for using artificial intelligence (AI) in real-time to assess the difficulty level of a patient being scanned and assigning an objective scanning difficulty level (SDL) to the patient to help inform medical personnel and educators of the difficulty of scanning said patient.

### TECHNICAL FIELD

The subject matter disclosed herein is generally directed to systems and methods for using artificial intelligence (AI) in real-time to assess the difficulty level of a patient being scanned and assigning an objective scanning difficulty level (SDL) to the patient to help inform medical personnel and educators of the difficulty of scanning said patient.

### BACKGROUND

There has been tremendous growth in the use of medical ultrasound in the past two decades. The development of portable ultrasound systems has expanded ultrasound users beyond the traditional users of radiologists, cardiologists, obstetricians, and sonographers to include almost every physician specialty and subspecialty as well as physician extenders like nurse practitioners and physician assistants. In addition, ultrasound is becoming an important component of healthcare education in medical schools, nursing schools, physician assistant programs, medical residency training, and other healthcare provider education.

Ultrasonography is a medical diagnostic imaging modality that is very operator-dependent and requires expert skill to acquire high quality ultrasound images to be used in making many important diagnostic and therapeutic medical decisions. This operator dependence is very different from other imaging modalities like computer tomography (CT) and magnetic resonance imaging (MRI) which are highly standardized with mostly automated imaging protocols. Ultrasound requires the operator to manually manipulate the ultrasound probe and adjust multiple machine parameters like the ultrasound wave frequency, focus, gain and depth or implementing harmonics to acquire quality ultrasound images.

In addition, every patient has unique anatomical features of the target ultrasound structure such as the heart and physical characteristics of the body tissue and other substances such as air in the ultrasound wave path between the ultrasound probe and the target structure. The ultrasound waves interact with all the material in the path as well as the target structure to create a spectrum of scanning difficulty levels for acquiring quality ultrasound images. This spectrum can vary from a “very easy” to scan patient to a “very difficult” to scan patient and occasionally even a patient in which quality ultrasound images simply cannot be attained and a different imaging modality such as CT must be used.

The variability of scanning difficulty from patient to patient can be due to difficulty factors such as the degree of subcutaneous fat just beneath the skin that the waves must travel through to reach the target structure, the depth in the body of the target structure, the size and three dimensional orientation of the target structure in the body, the size and location of any abnormality within the target structure, and characteristics of the various tissue interfaces between the probe and target structure, especially those involving air and bone. Air and bone are very strong reflectors of ultrasound waves and can interfere with the waves reaching the target structure.

Other factors that can affect the acquisition of quality images include abnormal or diseased tissue in the ultrasound path to the target structure, such as residual air in the lungs due to emphysema, calcification of tissue from inflammatory disease processes, and atherosclerosis of blood vessels. Because of air-filled blebs in the lungs, as a result of severe emphysema, ultrasound scanning of the heart in a patient with emphysema can be very difficult or even impossible using the standard transthoracic ultrasound cardiac views.

In addition, there are everyday biological activities that can significantly affect the ability to scan certain organs. For example, it can be very difficult to obtain quality images of structures in the abdomen such as the liver, gallbladder, spleen, pancreas, and aorta if the patient has eaten recently causing considerable bowel gas in the intestines, which like air limits the uniform penetration of the ultrasound waves to the target structures. Eating can also stimulate contraction of the gallbladder resulting in a much smaller gallbladder making adequate visual assessment of the gallbladder more difficult.

Considering the broad spectrum of patient scanning difficulty level and the operator-dependent nature of ultrasound, it would be extremely useful to have an objective measure of a patient's scanning difficulty level (SDL) for educational and medical practice activities. Accordingly, it is an object of the present disclosure to systematically and objectively assess the variables that impact the difficulty level of scanning in a specific patient. Knowing an individual patient's scanning difficulty level would assist in teaching ultrasound and in the assessment of ultrasound competency at both the trainee level, as well as the practicing ultrasound operator level across a wide spectrum of patient difficulty levels as is typically seen in medical practice. It would also allow more efficient and effective matching of the level of competency of the ultrasound operator and scanning difficultly level of the patient that could significantly improve practice workflow and the quality of patient assessment.

Furthermore, information learned in AI assessment of the SDL of a particular patient can then be used for auto-control of ultrasound parameters such as depth and gain to assist in the capture of higher quality images. This auto-control approach would enhance ease of use of the ultrasound device and improve quality of images beyond the presently used “preset” of parameters which are generally based on “average” patient characteristics. This more personalized auto-control approach could be applied to enhance image quality across multiple scanning scenarios including health professionals scanning, patient self-scanning, robotic scanning, and image acquisition from patient wearables with ultrasound capability. In addition, from an instructional perspective, an objective assessment of difficulty level could be applied to educational methods of learning ultrasound not involving actual scanning of real patients such as ultrasound simulation and gamification of ultrasound learning.

Citation or identification of any document in this application is not an admission that such a document is available as prior art to the present disclosure.

### SUMMARY

The above objectives are accomplished according to the present disclosure by providing a method for determining a patient's ultrasound scanning difficulty level (SDL). The method may include scanning the patient in at least one view using an ultrasound device to obtain an ultrasound scan image of the patient, employing at least one artificial intelligence in real time, which has been trained to identify and quantify the at least one ultrasound scan image obtained from the patient to: analyze the ultrasound scan image of the patient to, assess an ultrasound scanning difficulty level of the patient based on at least one patient physical characteristic; and assign a scanning difficulty level (SDL) to the patient. Further, the at least one artificial intelligence may auto control at least one parameter of the ultrasound device. Yet again, the at least one parameter of the ultrasound device auto controlled by the at least one artificial intelligence may be a gain and/or a scanning depth of the ultrasound device. Still yet, the SDL for the patient may be scored on a scanning difficulty scale. Further again, the scanning difficulty scale may comprise assigning at least one value to a level of patient scanning difficulty in order to assign the SDL for the patient. Still moreover, the assigned value may comprise a scale of values ranging between a lowest value indicating no patient scanning difficulty and a highest value indicating a highest patient scanning difficulty. Yet again, the at least one patient physical characteristic may comprise patient size, degree of body fat on the patient, tissue interfaces within the patient, disease processes, tissue calcification within the patient, quantity of gas within the patient, quantity of urine within the patient, presence of ultrasound artifacts obtained from examining the patient, target organ size, and/or abnormality of a target organ. Again, the method may combine the assigned SDL with at least one ultrasound image quality assessment tool. Even further, combining the assigned SDL with the at least one ultrasound image quality assessment tool may be used assess an ultrasound operator using the ultrasound device for at least one SDL. Even further, the method may include utilizing the assigned SDL to adjust the ultrasound device to establish at least one preset function setting for subsequent ultrasound examinations of the patient.

In a further embodiment, the current disclosure provides a system for determining a patient's ultrasound scanning difficulty level (SDL). The system may include an ultrasound device configured for scanning the patient in at least one view to obtain an ultrasound scan image of the patient, at least one artificial intelligence system, which has been configured to identify and quantify the at least one ultrasound scan image obtained from the patient to: analyze the ultrasound scan image of the patient to: assess an ultrasound scanning difficulty level of the patient based on at least one patient physical characteristic; assign a scanning difficulty level (SDL) to the patient; and wherein the artificial intelligence adjusts at least one ultrasound device ultrasound scanning parameter, without user interaction, to enhance image acquisition based on the assigned SDL for the patient. Further, the at least one ultrasound device ultrasound scanning parameter that may be controlled by the at least one artificial intelligence may be a gain and/or a scanning depth of the ultrasound device. Still yet, the SDL for the patient may be scored on a scanning difficulty scale. Further again, the scanning difficulty scale may comprise assigning at least one value to a level of patient scanning difficulty in order to assign the SDL for the patient. Again still, the assigned values may comprise a scale of values ranging between a lowest value indicating no patient scanning difficulty and a highest value indicating a highest patient scanning difficulty. Further again, the at least one patient physical characteristic may comprise patient size, degree of body fat on the patient, tissue interfaces within the patient, disease processes, tissue calcification within the patient, quantity of gas within the patient, quantity of urine within the patient, presence of ultrasound artifacts obtained from examining the patient, target organ size, and/or abnormality of a target organ. Even further yet, the system may combine the assigned SDL with at least one ultrasound image quality assessment tool. Still yet, the system may include combining the assigned SDL with the at least one ultrasound image quality assessment tool to assess an ultrasound operator using the ultrasound device for at least one SDL. Even further, the system may utilize the assigned SDL to adjust the ultrasound device to establish at least one preset function setting for subsequent ultrasound examinations of the patient.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present disclosure will be obtained by reference to the following detailed description that sets forth illustrative embodiments, in which the principles of the disclosure may be utilized, and the accompanying drawings of which:

FIG. 1 shows incomplete liver and kidney ultrasound images due to shadowing obstructions from bone (rib) and air (in the intestines).

FIG. 2 shows a diagram showing artificial intelligence development for assessment of scanning difficulty level.

FIG. 3 shows one embodiment of an AI apparatus of the current disclosure.

FIG. 4 shows a block diagram illustrating one embodiment of an AI server of the current disclosure.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless specifically stated, terms and phrases used in this document, and variations thereof, unless otherwise expressly stated, should be construed as open ended as opposed to limiting. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise.

Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used interchangeably herein, the terms “sufficient” and “effective,” can refer to an amount (e.g. mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

All patents, patent applications, published applications, and publications, databases, websites and other published materials cited herein are hereby incorporated by reference to the same extent as though each individual publication, published patent document, or patent application was specifically and individually indicated as being incorporated by reference.

### KITS

Any of the systems described herein can be presented as a combination kit. As used herein, the terms “combination kit” or “kit of parts” refers to the compounds, compositions, tools, and any additional components that are used to package, sell, market, deliver, and/or administer the combination of elements or a single element, such as a testing or ultrasound system, contained therein. Such additional components include, but are not limited to, packaging, syringes, blister packages, bottles, and the like. When one or more of the compounds, compositions, tools, and any additional components described herein or a combination thereof (e.g., machinery, medical implements, scanning devices, etc. contained in the kit are administered simultaneously, the combination kit can contain the compounds, compositions, tools, and any additional components together or separately. The separate kit components can be contained in a single package or in separate packages within the kit.

In some embodiments, the combination kit also includes instructions printed on or otherwise contained in a tangible medium of expression. The instructions can provide information regarding the content and usage of the kit, safety information regarding the contents, indications for use, and/or recommended treatment regimen(s) for the system and its component devices contained therein. In some embodiments, the instructions can provide directions and protocols for administering the system described herein to a subject in need thereof.

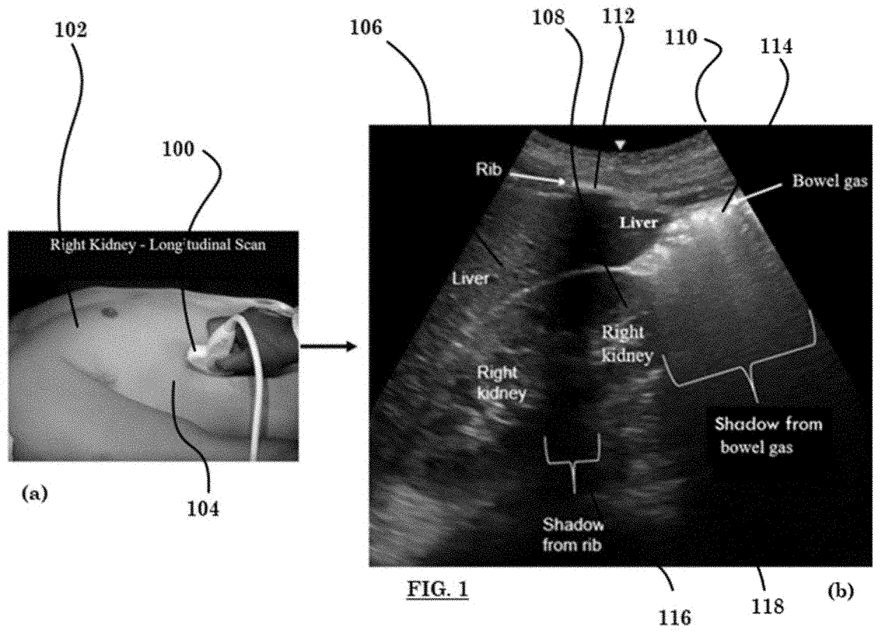

It is the intent of the present disclosure to use artificial intelligence (AI) in real-time to assess the difficulty level of the patient being scanned and assign an objective scanning difficulty level (SDL). As used herein “objective” refers to expressing ultrasound conditions while minimalizing subjective influences, such as personal feelings, prejudices, interpretations, etc., often introduced to ultrasound scanning techniques by ultrasound operators. As FIG. 1 shows, ultrasounds are complex data sources requiring nuanced understanding of what is being displayed in order to obtain accurate diagnoses. FIG. 1 at (a) shows an ultrasound probe 100 positioned on a patient 102 for the standard view of the longitudinal scan of the right kidney 104. As seen in FIG. 1 at (b), for patient 102, probe 100 at probe position 104 via scanning window 110 displays incomplete liver 106 and kidney 108 ultrasound images due to shadowing obstructions from bone 112 (rib) and air or gas 114 (in the intestines). The incomplete nature of the image shown by scanning window 110 shows “shadows” or dark areas in the scanning field such as shadow from rib 116 and shadow from bowel gas 118. These shadows block from view the underlying organs and prevent obtaining a clear ultrasound view of same.

FIG. 1 at (b) shows an example of how the presence of bone and bowel gas in the path of the ultrasound wave from the probe placed in the standard surface location or scanning window for the longitudinal view of the right kidney in this patient can create shadowing that limits the assessment of the liver and right kidney. Thus, this shadowing would contribute to a higher scanning difficulty level which would be a significant challenge to a novice scanner. However, an experienced (and more competent) scanner would have the knowledge and skill to, at least, partially overcome the challenge. To avoid the bone shadow produced by the rib the experienced scanner would ask the patient to take a deep breath and hold it, bringing the liver and kidney below the level of the ribs. The more competent scanner would also know to apply more pressure on the probe to move some of the intestinal air out of the ultrasound wave path to the target organs resulting in better ultrasound images. Much is known about the interaction of ultrasound waves and the human body in ultrasonography that can affect image quality. This information will be used in addition to expert opinion of experienced ultrasound operators to create initial algorithm rules to begin training and testing in the AI development process.

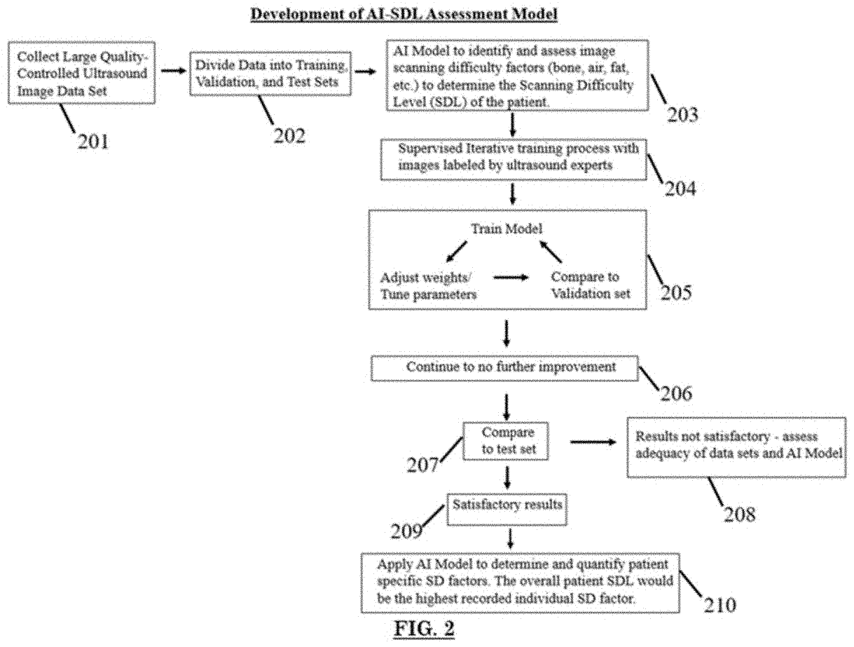

It is anticipated that multiple approaches to development of the final software product will be explored on a large and varied population of patients and images. This will include but not limited to supervised and unsupervised training and multiple layers of deep learning and neural networks. Such an example is shown in FIG. 2 that includes collection of a large quality-controlled ultrasound image data set 201 that would be divided into training, validation, and test sets 202. The AI model could be trained to identify and quantify image scanning difficulty factors such as bone, air, fat, etc., see step 203, using an iterative supervised training process with images labeled by ultrasound experts 204. Each iteration would be compared to the validation set 205 and continued until there was no further improvement 206. Comparison would then be with the test set 207 and, if results are not satisfactory, assessment of the adequacy of the datasets and AI methodology would be made 208. If results are satisfactory 209, the model would be ready for application. Each scanning difficulty factor would be identified and scored as 0 if no difficulty is identified, 1 for mild difficulty, 2 for moderate difficulty, 3 for extreme difficulty and 4 for a difficulty level that does not allow an acceptable image to be acquired from the ultrasound window being assessed. While a 0 to 4 scale is shown, the current disclosure is not so limited. Any scale may be used that establishes a range of values starting (or ending) with a lowest setting indicating no patient scanning difficulty to a highest value indicating a the most difficult or highest patient scanning difficulty The SDL for the patient would be the highest score of all the individual difficulty factors as that would be the overall limiting scanning factor for the patient 210. For instance, the presence of fat, air, bone, or any other factor affecting the quality of ultrasound detection may be employed to arrive an SDL value for a patient. The SDL-AI software can be developed and combined with a number of network architectures and machine learning libraries such as GoogleNet, ResNet, VGGNet, Inception, TensorFlow, Caffe and others of which many are open-source.

Some of the factors known to affect the ability to obtain quality ultrasound images include: size of the patient and distance of the target structures from the ultrasound probe; degree of body fat that must be penetrated between the probe and target structures; tissue interfaces and other structures in the ultrasound wave path to the target structures; the plane of the target structures relative to the angle of the beam possible at the body surface; degree and rate of movement of the target structure while scanning such as a rapidly beating heart and movement of the entire heart with respirations; disease processes affecting the target tissue and tissue between the probe and the target tissue such as air in lung tissue; bone and various forms of tissue calcification; bowel gas from eating and normal physiological processes; quantity of urine in the bladder when performing pelvic ultrasound; quality of available ultrasound windows for scanning; presence and specific causes of ultrasound artifacts (image illusions) that appear on the image display screen; target organ size; and size of an abnormality in the target organ. The contribution of subcutaneous individual difficulty or image limiting factors can be assessed by a standardized method and scale for each factor. For example, the contribution of fat for an abdominal ultrasound scan of the aorta could be determined by measuring the thickness of subcutaneous fat at the location of the ultrasound probe placement on the patient's abdomen when obtaining the scan. The highest individual scanning difficulty factors can then be combined for AI determination of the overall Scanning Difficulty Level for the specific patient as that would be the overall limiting factor.

The current disclosure provides various novel features including: artificial intelligence used to develop a spectrum of objective ultrasound scan difficulty levels (SDL) based on a wide variety of patient physical characteristics that affect ultrasound waves and resulting images and artificial intelligence will be used in real-time ultrasound scanning to assign an SDL to an individual patient. Further, the combined SDL-AI will allow assessment of levels of competency of an ultrasound operator across a spectrum of patient difficulty levels. Knowing the SDL of the patient being scanned combined with AI and/or expert opinion grading of image quality obtained by the learner or ultrasound practitioner can produce an assessment of an operator's ultrasound skill or competency. There are ultrasound image quality assessment tools already available on some ultrasound machines, but these are not interpreted in the context of the difficulty level of the patient being scanned. The SDL-AI software can be combined with available AI automated image grading software, such as VDMX, Syphon, Synposis, CinemaNet, Colourlab AI, etc., and/or expert opinion grading image quality to produce an assessment of an operator's ultrasound skill or competency. The SDL-AI system can also be used to establish a level of confidence of automated ultrasound image grading software by putting the image in the context of the patient's SDL.

Artificial intelligence refers to the field of studying artificial intelligence or methodology for making artificial intelligence, and machine learning refers to the field of defining various issues dealt with in the field of artificial intelligence and studying methodology for solving the various issues. Machine learning is defined as an algorithm that enhances the performance of a certain task through a steady experience with the certain task.

An artificial neural network (ANN) is a model used in machine learning and may mean a whole model of problem-solving ability which is composed of artificial neurons (nodes) that form a network by synaptic connections. The artificial neural network can be defined by a connection pattern between neurons in different layers, a learning process for updating model parameters, and an activation function for generating an output value.

The artificial neural network may include an input layer, an output layer, and optionally one or more hidden layers. Each layer includes one or more neurons, and the artificial neural network may include a synapse that links neurons to neurons. In the artificial neural network, each neuron may output the function value of the activation function for input signals, weights, and deflections input through the synapse.

Model parameters refer to parameters determined through learning and include a weight value of synaptic connection and deflection of neurons. A hyperparameter means a parameter to be set in the machine learning algorithm before learning, and includes a learning rate, a repetition number, a mini batch size, and an initialization function.

The purpose of the learning of the artificial neural network may be to determine the model parameters that minimize a loss function, such as minimize poor ultrasound scanning techniques and/or show how to improve same. The loss function may be used as an index to determine optimal model parameters in the learning process of the artificial neural network.

Machine learning may be classified into supervised learning, unsupervised learning, and reinforcement learning according to a learning method.

The supervised learning may refer to a method of learning an artificial neural network in a state in which a label for training data is given, and the label may mean the correct answer (or result value) that the artificial neural network must infer when the training data is input to the artificial neural network. The unsupervised learning may refer to a method of learning an artificial neural network in a state in which a label for training data is not given. The reinforcement learning may refer to a learning method in which an agent defined in a certain environment learns to select a behavior or a behavior sequence that maximizes cumulative compensation in each state.

The current disclosure may provide neural net systems that may connect to, be integrated in, and be accessible by a processor, computer, cloud-based system, and/or platform for enabling intelligent transactions including ones involving expert systems, self-organization, machine learning, artificial intelligence and including neural net systems trained for pattern recognition, for classification of one or more parameters, characteristics, or phenomena, for support of autonomous control, and other purposes in accordance with embodiments of the present disclosure. Indeed, the AI associated with the current disclosure may include removing an input that is the source of the error, such a poor user angle, poor setting choice, insufficient pressure, etc., reconfiguring a set of nodes of the artificial intelligence system, reconfiguring a set of weights of the artificial intelligence system, reconfiguring a set of outputs of the artificial intelligence system, reconfiguring a processing flow within the artificial intelligence system, and augmenting the set of inputs to the artificial intelligence system, and change the settings on the ultrasound device to “override” the poor user input and/or improve same.

Further, in some embodiments, an artificial intelligence system may be trained to perform an action selected from among determining an architecture for a ultrasound system, reporting on a status, reporting on an event, reporting on a context, reporting on a condition, determining a model, configuring a model, populating a model, designing a system, designing a process, designing an apparatus, engineering a system, engineering a device, engineering a process, engineering a product, maintaining a system, maintaining a device, maintaining a process, maintaining a network, maintaining a computational resource, maintaining equipment, maintaining hardware, repairing a system, repairing a device, repairing a process, repairing a network, repairing a computational resource, repairing equipment, repairing hardware, assembling a system, assembling a device, assembling a process, assembling a network, assembling a computational resource, assembling equipment, assembling hardware, setting a price, physically securing a system, physically securing a device, physically securing a process, physically securing a network, physically securing a computational resource, physically securing equipment, physically securing hardware, cyber-securing a system, cyber-securing a device, cyber-securing a process, cyber-securing a network, cyber-securing a computational resource, cyber-securing equipment, cyber-securing hardware, detecting a threat, detecting a fault, tuning a system, tuning a device, tuning a process, tuning a network, tuning a computational resource, tuning equipment, tuning hardware, optimizing a system, optimizing a device, optimizing a process, optimizing a network, optimizing a computational resource, optimizing equipment, optimizing hardware, monitoring a system, monitoring a device, monitoring a process, monitoring a network, monitoring a computational resource, monitoring equipment, monitoring hardware, configuring a system, configuring a device, configuring a process, configuring a network, configuring a computational resource, configuring equipment, and configuring hardware.

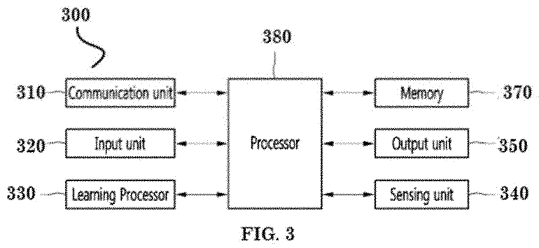

Referring to FIG. 3, the AI apparatus 300 may include a communication unit 310, an input unit 320, a learning processor 330, a sensing unit 340, an output unit 350, a memory 370, and a processor 380.

The communication unit 310 may transmit and receive data to and from external devices such as other devices 300 a to 300 e and the AI server by using wire/wireless communication technology. For example, the communication unit 310 may transmit and receive sensor information, a user input, a learning model, and a control signal to and from external devices.

The communication technology used by the communication unit 310 includes GSM (Global System for Mobile communication), CDMA (Code Division Multi Access), LTE (Long Term Evolution), 5G, WLAN (Wireless LAN), Wi-Fi (Wireless-Fidelity), Bluetooth™, RFID (Radio Frequency Identification), Infrared Data Association (IrDA), ZigBee, NFC (Near Field Communication), and the like.

The input unit 320 may acquire various kinds of data.

Here, the input unit 320 may include a camera for inputting a video signal, a microphone for receiving an audio signal, and a user input unit for receiving information from a user or from an ultrasound device. The camera or the microphone or the ultrasound device may be treated as a sensor, and the signal acquired from the camera or the microphone or the ultrasound device may be referred to as sensing data or sensor information.

The input unit 320 may acquire a training data for model learning and an input data to be used when an output is acquired by using learning model. The input unit 320 may acquire raw input data. Here, the processor 380 or the learning processor 330 may extract an input feature by preprocessing the input data.

The learning processor 330 may learn a model composed of an artificial neural network by using training data. The learned artificial neural network may be referred to as a learning model. The learning model may be used to an infer result value for new input data rather than training data, and the inferred value may be used as a basis for determination to perform a certain operation.

The learning processor 330 may perform AI processing together with a learning processor of an AI server, not shown. The learning processor 330 may include a memory integrated or implemented in the AI apparatus 300. Alternatively, the learning processor 330 may be implemented by using the memory 370, an external memory directly connected to the AI apparatus 300, or a memory held in an external device.

The sensing unit 340 may acquire at least one of internal information about the AI apparatus 300, ambient environment information about the AI apparatus 300, and user information by using various sensors, such as the ultrasound device, camera, microphone, etc.

Examples of the sensors included in the sensing unit 340 may include those common to an ultrasound device as well as a proximity sensor, an illuminance sensor, an acceleration sensor, a magnetic sensor, a gyro sensor, an inertial sensor, an RGB sensor, an IR sensor, a fingerprint recognition sensor, an ultrasonic sensor, an optical sensor, a microphone, a lidar, and a radar.

The output unit 350 may generate an output related to a visual sense, an auditory sense, or a haptic sense. Here, the output unit 350 may include a display for outputting time information, displaying ultrasound images and corrections/changes to ultrasound device settings, a speaker for outputting auditory information, and a haptic module for outputting haptic information. Memory 370 may store data that supports various functions of the AI apparatus 300. For example, memory 370 may store input data acquired by the input unit 320, training data, a learning model, a learning history, and the like. Processor 380 may determine at least one executable operation of the AI apparatus 300 based on information determined or generated by using a data analysis algorithm or a machine learning algorithm. The processor 380 may control the components of the AT apparatus 300 to execute the determined operation. To this end, the processor 380 may request, search, receive, or utilize data of the learning processor 330 or the memory 370. The processor 380 may control the components of the AT apparatus 300 to execute the predicted operation or the operation determined to be desirable among the at least one executable operation. When the connection of an external device is required to perform the determined operation, the processor 380 may generate a control signal for controlling the external device, such as an ultrasound scanning device or accoutrement technologies, and may transmit the generated control signal to the external device. Processor 380 may acquire intention information for the user input and may determine the user's requirements based on the acquired intention information.

The processor 380 may acquire the intention information corresponding to the user input by using at least one of a speech to text (STT) engine for converting speech input into a text string or a natural language processing (NLP) engine for acquiring intention information of a natural language.

At least one of the STT engine or the NLP engine may be configured as an artificial neural network, at least part of which is learned according to the machine learning algorithm. At least one of the STT engine or the NLP engine may be learned by the learning processor 330, may be learned by the learning processor of the AI server, not shown, or may be learned by their distributed processing.

Processor 380 may collect history information including the operation contents of the AT apparatus 300 or the user's feedback on the operation and may store the collected history information in the memory 370 or the learning processor 330 or transmit the collected history information to the external device such as an AT server. The collected history information may be used to update the learning model.

The processor 380 may control at least part of the components of AI apparatus 300 so as to drive an application program stored in memory 370. Furthermore, the processor 380 may operate two or more of the components included in the AI apparatus 300 in combination so as to drive the application program.

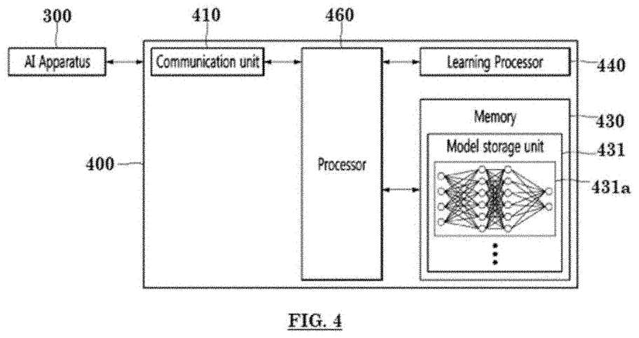

FIG. 4 is a block diagram illustrating an AI server 400 according to an embodiment. Referring to FIG. 4, AI server 400 may refer to a device that learns an artificial neural network by using a machine learning algorithm or uses a learned artificial neural network. AI server 400 may include a plurality of servers to perform distributed processing, or may be defined as a 5G network. Here, AI server 400 may be included as a partial configuration of the AI apparatus 300, and may perform at least part of the AI processing together AI server 400 may include a communication unit 410, a memory 430, a learning processor 440, a processor 460, and the like. Communication unit 410 can transmit and receive data to and from an external device such as the AI apparatus 300. Memory 430 may include a model storage 431. The model storage 431 may store a learning or learned model (or an artificial neural network 431a) through the learning processor 440. Learning processor 440 may learn the artificial neural network 431a by using the training data. The learning model may be used in a state of being mounted on the AI server 400 of the artificial neural network, or may be used in a state of being mounted on an external device such as the AI apparatus 300.

The learning model may be implemented in hardware, software, or a combination of hardware and software. If all or part of the learning models are implemented in software, one or more instructions that constitute the learning model may be stored in memory 430. Processor 460 may infer the result value for new input data by using the learning model and may generate a response or a control command based on the inferred result value.

The SDL-AI can enhance the accuracy and confidence level of automated ultrasound image grading software by putting the image in the context of the patient's SDL. Further, the SDL-AI can estimate the time to complete an ultrasound examination based on the SDL of the patient from previous ultrasound examinations. This estimated time to scan a patient can be used to improve practice workflow by more accurately estimating the time to perform an ultrasound follow-up examination. Recorded scan time to scan from previous ultrasound examinations can be added to the patient's SDL-AT to further enhance the accuracy of the estimated time to scan for scheduling future ultrasound examinations, especially if matched with the competency level of the previous ultrasound operators who perform the scans.

The SDL of a live model used in a practical testing method of ultrasound competency such as an objective structured clinical examination (OSCE) for ultrasound would help ensure the difficulty level of the model used for the examination would be consistent with the expected level of competency of those taking the examination and not too hard or too easy. Further, a SDL may be used to standardize OSCE exams for various learners and levels of competency. The SDL-AT data collected across many patients can be used in creating more accurate ultrasound simulated cases for ultrasound simulators to enhance training and assessment across a wide range of scanning difficulty levels. The SDL-AT data collected across many patients will also allow creation of more realistic gamification cases of ultrasound scanning. Adding scanning difficulty factors to simulation and gamification would be a novel introduction to enhance ultrasound learning. At present simulation and gamification have focused on identifying important structures and pathology but not in the context of scanning difficulty factors which need to be identified and minimized to enhance scanning competency.

SDL applications include: assessing competency of ultrasound learners and practitioners by grading their images in the context of difficulty to scan; facilitating competency-based education models such that the ability to scan more difficult patients corresponds to increased skill and competency; determining clinical ultrasound credentialing and clinical privileging for practitioners; developing assessment methods of ultrasound operators for new ultrasound applications as they become available; assessing competency level from scanning difficulty levels assessment data to assist with decisions on the level of supervision needed for new learners and relatively inexperienced ultrasound practitioners; following progression of skill through ultrasound milestones toward competency for learners; and using established difficulty factors and newly discovered difficulty factors from the SDL-AI data to enhance ultrasound curricula for all learners and medical practitioners.

Scanning solutions as determined by ultrasound expert consensus opinion and/or AI to overcome the variety of scanning difficulties can be made available either automatically or on-demand to ultrasound operators as the SDL-AI identifies a difficulty in the patient being scanned. The solutions can be built into the software of the ultrasound system to automatically adjust certain features of the system such as harmonics once a scanning difficulty is identified. Unique approaches based on recognized scanning difficulty factors such as significant intestinal gas or a rib shadow could instruct the ultrasound operator to try applying slightly more probe pressure or asking the patient to take a deep breath and hold it respectively to minimize these difficulty scanning factors. Such automated instructions are presently not available to ultrasound operators and learners for difficulty scanning factors. Similarly, the SDL-AI may be added to robotic ultrasonography, including the degree of pressure on the body surface with the probe which can help overcome difficulties such as abdominal gas or significant subcutaneous abdominal fat in the ultrasound path. SDL-AI can be used for real-time self-directed learning of ultrasound as immediate feedback on specific patient scanning difficulties could be provided along with on-demand scanning advice to address the scanning difficulties as needed.

The current disclosure may also differentiate difficulty factors that are relatively stable in the patient such as organ location and calcification of structures from short-lived temporal factors that are associated with scanning conditions at the time of the scan such as the degree of bowel gas due to eating prior to the scanning session. In the case of temporary factors, the best workflow approach may be to simply reschedule the patient for a later date, if possible, with specific instructions to avoid food or chewing gum for several hours prior to the abdominal ultrasound examination. The disclosure also provides the possibility to combine patent scanning difficulty level-AI with AI software to grade the quality of an image to advance measurement of skill and competency of learners and practitioners and provide immediate feedback to the operator. Further, based on identification of patient specific scanning difficulties with SDL-AI from previous ultrasound scans, new ultrasound examinations can be “personalized” with specific machine settings and available expert advice to eliminate or minimize the known scanning difficulties of the patient.

Provided herein are methods, systems, components and other elements for an ultrasound information technology system that may include: a cloud-based management platform with a micro-services architecture, a set of interfaces, network connectivity facilities, adaptive intelligence facilities, data storage facilities, and monitoring facilities that are coordinated for monitoring and management of a set of value chain network entities; a set of applications for enabling an enterprise to manage a set of ultrasound network entities from a point of origin to a point of use; and a set of microservices layers including an application layer supporting at least one ultrasound application and at least one demand management application, wherein the microservice layers include a process automation layer that uses information collected by a data collection layer and a set of outcomes and activities involving the applications of the application layer to automate a set of actions for at least a subset of the applications.

In embodiments, methods and systems described herein that involve an expert system or self-organization capability may use a physical neural network where one or more hardware elements may be used to perform or simulate neural behavior. One or more hardware nodes may be configured to stream output data resulting from the activity of the neural net. Hardware nodes, which may comprise one or more chips, microprocessors, integrated circuits, programmable logic controllers, application-specific integrated circuits, field-programmable gate arrays, or the like, may be provided to optimize the speed, input/output efficiency, energy efficiency, signal to noise ratio, or other parameter of some part of a neural net of any of the types described herein. Hardware nodes may include hardware for acceleration of calculations (such as dedicated processors for performing basic or more sophisticated calculations on input data to provide outputs, dedicated processors for filtering or compressing data, dedicated processors for de-compressing data, dedicated processors for compression of specific file or data types (e.g., for handling image data, video streams, acoustic signals, vibration data, thermal images, heat maps, or the like), and the like. A physical neural network may be embodied in a data collector, edge intelligence system, adaptive intelligent system, mobile data collector, IoT monitoring system, or other system described herein, including one that may be reconfigured by switching or routing inputs in varying configurations, such as to provide different neural net configurations within the system for handling different types of inputs (with the switching and configuration optionally under control of an expert system, which may include a software-based neural net located on the data collector or remotely). A physical, or at least partially physical, neural network may include physical hardware nodes located in a storage system, such as for storing data within machine, a product, or the like, such as for accelerating input/output functions to one or more storage elements that supply data to or take data from the neural net. A physical, or at least partially physical, neural network may include physical hardware nodes located in a network, such as for transmitting data within, to or from an environment, such as for accelerating input/output functions to one or more network nodes in the net, accelerating relay functions, or the like. In embodiments, of a physical neural network, an electrically adjustable resistance material may be used for emulating the function of a neural synapse. In embodiments, the physical hardware emulates the neurons, and software emulates the neural network between the neurons. In embodiments, neural networks complement conventional algorithmic computers. They may be trained to perform appropriate functions without the need for any instructions, such as classification functions, optimization functions, pattern recognition functions, control functions, selection functions, evolution functions, and others.

Indeed the integration of AI with the present disclosure can allow the AI to adjust settings on the ultrasound device automatically, without user input. This can include adjusting sonographic imaging modes, such as conventional imaging, compound imaging, and tissue harmonic imaging, as known to those of skill in the art. The AI can also adjust the depth, frequency, focusing, gain, and Doppler of the ultrasound device, real time display, time gain compensation, focal zone, gray scale, dynamic range, persistence, frame rate, pulse repetition frequency, color flow, wall filter, and/or sample gate, all without user input or the user adjusting these parameters, based on the AI determining the preferred settings for the patient vis-à-vis the patient's SDL.

Definitions

Real Time—(B Mode/2D) Ultrasound instrumentation that allows the image to be displayed many times per second to achieve a “real-time” image of anatomic structures and their motion patterns

Gain—Measure of the strength of the ultrasound signal; overall gain amplifies all signals by a constant factor regardless of the depth

TGC—Time Gain Compensation; Ability to compensate for the attenuation of the transmittal beam as the sound wave travels through tissue in the body. The goal of TGC is to make the entire image look evenly lit from top to bottom

Focal Zone—The region over which the effective width of the sound beam is within some measure of its width at the local distance

Frequency—Number of cycles per second that a periodic event or function undergoes; number of cycles completed per unit of time; the frequency of a sound wave is determined by the number of oscillations per second of the vibrating source

Gray Scale—A series of shades from white to black. B-Mode scanning technique that permits the brightness of the B-Mode dots to be displayed in various shades of gray to represent different echo amplitudes

Dynamic Range—Ratio of the largest to the smallest signals that an instrument or component of an instrument can respond to without distortion. It controls the contrast on the ultrasound image making an image look either very gray or very black and white

Persistence—is a type of temporal smoothing used in both gray scale and color Doppler imaging. Successive frames are averaged as they are displayed to reduce the variations in the image between frames, hence lowering the temporal resolution of the image

Frame Rate—Rate at which images are updated on the display; dependent on frequency of the transducer and depth selection

PRF—Pulse Repetition Frequency (scale); in pulse echo instruments, it is the number of pulses launched per second by the transducer

PW Doppler—Pulsed Wave Doppler; sound is transmitted and received intermittently with one transducer. PW allows us to measure blood velocities at a single point, or within a small window of space

Color Flow—Ability to display blood flow in multiple colors depending on the velocity, direction of flow and extent of turbulence

CW Doppler—Continuous wave Doppler; one transducer continuously transmits sound and one continuously receives sound; used in high velocity flow patterns

Wall Filter—a high-pass filter usually employed to remove the wall component from the blood flow signal

Doppler Angle—The angle that the reflector path makes with the ultrasound beam; the most accurate velocity is recorded when the beam is parallel to flow

Sample Gate—The sample site from which the signal is obtained with pulsed Doppler

Various modifications and variations of the described methods, pharmaceutical compositions, and kits of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

What is claimed is:

1. A method for determining a patient's ultrasound scanning difficulty level (SDL) comprising:
scanning the patient in at least one view using an ultrasound device to obtain an ultrasound scan image of the patient;
employing at least one artificial intelligence in real time, which has been trained to identify and quantify the at least one ultrasound scan image obtained from the patient to:
analyze the ultrasound scan image of the patient to:
assess an ultrasound scanning difficulty level of the patient based on at least one patient physical characteristic; and
assign a scanning difficulty level (SDL) to the patient.

2. The method of claim 1, wherein the at least one artificial intelligence auto controls at least one parameter of the ultrasound device.

3. The method of claim 2, wherein the at least one parameter of the ultrasound device auto controlled by the at least one artificial intelligence is a gain and/or a scanning depth of the ultrasound device.

4. The method of claim 1, wherein the SDL for the patient is scored on a scanning difficulty scale.

5. The method of claim 4, wherein the scanning difficulty scale comprises assigning at least one value to a level of patient scanning difficulty in order to assign the SDL for the patient.

6. The method of claim 5, wherein the assigned values comprise a scale of values ranging between a lowest value indicating no patient scanning difficulty and a highest value indicating a highest patient scanning difficulty.

7. The method of claim 1, further comprising wherein the at least one patient physical characteristic comprises patient size, degree of body fat on the patient, tissue interfaces within the patient, disease processes, tissue calcification within the patient, quantity of gas within the patient, quantity of urine within the patient, presence of ultrasound artifacts obtained from examining the patient, target organ size, and/or abnormality of a target organ.

8. The method of claim 1, further comprising combining the assigned SDL with at least one ultrasound image quality assessment tool.

9. The method of claim 8, wherein combining the assigned SDL with the at least one ultrasound image quality assessment tool to assess an ultrasound operator using the ultrasound device for at least one SDL.

10. The method of claim 1, further comprising utilizing the assigned SDL to adjust the ultrasound device to establish at least one preset function setting for subsequent ultrasound examinations of the patient.

11. A system for determining a patient's ultrasound scanning difficulty level (SDL) comprising:
an ultrasound device configured for scanning the patient in at least one view to obtain an ultrasound scan image of the patient;
at least one artificial intelligence system, which has been configured to identify and quantify the at least one ultrasound scan image obtained from the patient to:
analyze the ultrasound scan image of the patient to:
assess an ultrasound scanning difficulty level of the patient based on at least one patient physical characteristic;
assign a scanning difficulty level (SDL) to the patient; and
wherein the artificial intelligence adjusts at least one ultrasound device ultrasound scanning parameter, without user interaction, to enhance image acquisition based on the assigned SDL for the patient.

12. The system of claim 11, wherein the at least one ultrasound device ultrasound scanning parameter controlled by the at least one artificial intelligence is a gain and/or a scanning depth of the ultrasound device.

13. The system of claim 11, wherein the SDL for the patient is scored on a scanning difficulty scale.

14. The system of claim 13, wherein the scanning difficulty scale comprises assigning at least one value to a level of patient scanning difficulty in order to assign the SDL for the patient.

15. The system of claim 14, wherein the assigned values comprise a scale of values ranging between a lowest value indicating no patient scanning difficulty and a highest value indicating a highest patient scanning difficulty.

16. The system of claim 11, further comprising wherein the at least one patient physical characteristic comprises patient size, degree of body fat on the patient, tissue interfaces within the patient, disease processes, tissue calcification within the patient, quantity of gas within the patient, quantity of urine within the patient, presence of ultrasound artifacts obtained from examining the patient, target organ size, and/or abnormality of a target organ.

17. The system of claim 11, further comprising combining the assigned SDL with at least one ultrasound image quality assessment tool.

18. The system of claim 18, further comprising combining the assigned SDL with the at least one ultrasound image quality assessment tool to assess an ultrasound operator using the ultrasound device for at least one SDL.

19. The system of claim 11, further comprising utilizing the assigned SDL to adjust the ultrasound device to establish at least one preset function setting for subsequent ultrasound examinations of the patient.
